(19) United States
(12) Patent Application Publication
(10) Pub. No.: US 2006/0167292 A1
(43) Pub. Date: Jul. 27, 2006

(54) Process for preparing enantiomerically enriched 2-fluorocarboxylic esters

(76) Inventor: Arne Gerlach, Odenthal (DE)

Correspondence Address: LANXESS CORPORATION, 111 RIDC PARK WEST DRIVE, PITTSBURGH, PA, 15275-1112, US

(21) Appl. No.: 11/301,419

(22) Filed: Dec. 13, 2005

(30) Foreign Application Priority Data
Dec. 16, 2004 (DE) 10 2004 060 493.2

(51) Int. Cl. C07C 69/76; C07C 69/63

(52) U.S. Cl. 554/225; 560/227; 560/103

(57) ABSTRACT

The present invention relates to a process for preparing enantiomerically enriched 2-fluorocarboxylic esters, in particular methyl (R)-2-fluoropropionate, from (S)-2-sulphonyloxycarboxylic esters and potassium fluoride.

The present invention relates to a process for preparing enantiomerically enriched 2-fluorocarboxylic esters, in particular methyl (R)-2-fluoropropionate, from (S)-2-sulphonyloxycarboxylic esters and potassium fluoride.

Methyl (R)-2-fluoropropionate and other optically active 2-fluorocarboxylic acid derivatives are important synthetic units for the development of novel active ingredients (for example JP-A 2000178259) and ferroelectric liquid-crystalline phases (for example DE-A 38 36 855).

The literature describes several processes for preparing enantiomerically enriched 2-fluorocarboxylic esters, in particular methyl 2-fluoropropionate. DE-A 41 31 242 describes, for example, the preparation of optically active 2-fluorocarboxylic esters from (S)-2-sulphonyloxycarboxylic esters and potassium fluoride, in particular of methyl (R)-2-fluoropropionate from methyl (S)-2-mesyloxypropionate and potassium fluoride in formamide. To this end, first a solution of potassium fluoride in formamide is prepared and then the (S)-2-sulphonyloxycarboxylic ester is added to this heated potassium fluoride solution. However, the procedure described here has the following disadvantages:
1. In order to achieve the best results, it is necessary, for example in the preparation of methyl (R)-2-fluoropropionate, to use 4.0 equivalents of potassium fluoride based on methyl (S)-2-mesyloxypropionate. This large excess of potassium fluoride is disadvantageous from an industrial point of view, since 3.0 equivalents remain unutilized in this reaction and cannot be recovered after the end of the reaction.
2. The reaction time is additionally relatively long at 4 hours. Here too, a reduction would be desirable from an industrial point of view.
3. When methyl (S)-2-mesyloxypropionate having an optical purity of 97% is used in the reaction, the desired product, methyl (R)-2-fluoropropionate, is obtained only with an optical purity of 96%. During the reaction, 1% racemization thus occurs.

There is thus still a need for a process for preparing enantiomerically enriched 2-fluorocarboxylic esters, in particular methyl (R)-2-fluoropropionic esters, which does not have these disadvantages.

It is thus an object of the present invention to discover a process for preparing enantiomerically enriched 2-fluorocarboxylic esters, in particular methyl (R)-2-fluoropropionate, which leads to the desired product with minimum racemization and a small fluoride excess in a relatively short reaction time.

It has been found that, surprisingly, enantiomerically enriched 2-fluorocarboxylic esters, when at least one alkali metal fluoride or tetraalkylammonium fluoride in a suitable solvent is added to initially charged (S)-2-sulphonyloxycarboxylic esters, can be prepared with fewer than 4.0 equivalents of fluoride, a reaction time of fewer than 4 hours and with racemization distinctly below 1%.

The present invention therefore provides a process for preparing an enantiomerically enriched 2-fluorocarboxylic ester of the general formula (I) 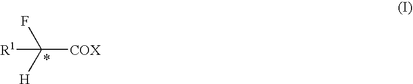
in which
R¹ is an optionally substituted C₁-C₁₈-alkyl radical, preferably an optionally substituted C₁-C₆-alkyl radical, an optionally substituted C₄-C₂₄-aryl radical, preferably an optionally substituted C₆-C₂₄-aryl radical, or an optionally substituted C₅-C₁₈-arylalkyl radical and
X is halogen, —NR²R³ or —OR⁴, in which R², R³ and R⁴ are each independently H or an optionally substituted C₁-C₁₈-alkyl radical, preferably an optionally substituted C₁-C₆-alkyl radical, an optionally substituted C₄-C₂₄-aryl radical, preferably an optionally substituted C₆-C₂₄-aryl radical, or an optionally substituted C₅-C₁₈-arylalkyl radical, or R² and R³ together are an optionally substituted C₁-C₁₈-alkylene radical, preferably an optionally substituted C₂-C₆-alkylene radical, an optionally substituted C₄-C₂₄-arylene radical, preferably an optionally substituted C₆-C₂₄-arylene radical, or an optionally substituted C₅-C₁₈-arylalkylene radical,
   by reacting an enantiomerically enriched sulphonic ester of the general formula
   (II) 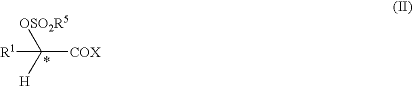
   in which
R⁵ is an optionally substituted C₁-C₁₈-alkyl radical, preferably an optionally substituted C₁-C₆-alkyl radical, an optionally substituted C₄-C₂₄-aryl radical, preferably an optionally substituted C₆-C₂₄-aryl radical, or an optionally substituted C₅-C₁₈-arylalkyl radical and
R¹ and X are each as defined for the general formula (I)
   with at least one alkali metal fluoride or tetraalkylammonium fluoride in a solvent comprising at least one carboxamide, characterized in that the sulphonic ester of the general formula (II) is initially charged and a mixture of at least one alkali metal fluoride or tetraalkylammonium fluoride and at least one carboxamide is subsequently added.

The enantiomerically enriched sulphonic ester of the general formula (II), also referred to below for short as sulphonic ester of the general formula (II), is initially charged preferably at a pressure of less than 1 bar, more preferably less than 500 mbar, most preferably less than 50 mbar.

Moreover, the sulphonic ester of the general formula (II) is initially charged preferably at a temperature of at least 50° C., more preferably of 50 to 100° C., most preferably of 70 to 85° C.

In preferred embodiments of the process according to the invention, the sulphonic ester of the general formula (II) is initially charged at a temperature of at least 50° C. and a pressure of less than 1 bar, preferably at a temperature of 50° C. to 100° C. and a pressure of less than 500 mbar, more preferably at a temperature of 70 to 85° C. and a pressure of less than 50 mbar.

The partial or full performance of the process according to the invention under a protective gas atmosphere, for example nitrogen or argon atmosphere, may be advantageous, but is not absolutely necessary.

Alkyl or alkylene are each independently a linear, cyclic, branched or unbranched alkyl or alkylene radical. The same applies to the nonaromatic moiety of an arylalkyl or arylalkylene radical.

C₁-C₆-Alkyl is, for example, methyl, ethyl, n-propyl, isopropyl, n-butyl, sec-butyl, tert-butyl, n-pentyl, 1-methylbutyl, 2-methylbutyl, 3-methylbutyl, neopentyl, 1-ethylpropyl, cyclohexyl, cyclopentyl, n-hexyl, 1,1-dimethylpropyl, 1,2-dimethylpropyl, 1,2-dimethylpropyl, 1-methypentyl, 2-methypentyl, 3-methylpentyl, 4-methylpentyl, 1,1-dimethylbutyl, 1,2-dimethylbutyl, 1,3-diemthylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl or 1-ethyl-2-methypropyl, and C₁-C₁₈-alkyl is additionally, for example, n-heptyl and n-octyl, pinacolyl, adamantyl, the isomeric menthyls, n-nonyl, n-decyl, n-dodecyl, n-tridecyl, n-tetradecyl, n-hexadecyl or n-octadecyl.

C₂-C₆-Alkylene is, for example, ethylene, n-propylene, isopropylene, n-butylene, n-pentylene, n-hexylene, and C₁-C₁₈-alkylene is additionally, for example, methylene, n-heptylene and n-octylene, n-nonylene, n-decylene, n-dodecylene, n-tridecylene, n-tetradecylene, n-hexadecylene or n-octadecylene.

Aryl is in each case independently an aromatic radical having 4 to 24 skeleton carbon atoms in which no, one, two or three skeleton carbon atoms per cycle, but at least one skeleton carbon atom in the entire molecule, may be substituted by heteroatoms selected from the group of nitrogen, sulphur or oxygen, but is preferably a carbocyclic aromatic radical having 6 to 24 skeleton carbon atoms. The same also applies to the aryl moiety of an arylalkyl or arylalkylene radical.

Examples of C₆-C₂₄-aryl are phenyl, o-, p-, m-tolyl, naphthyl, phenanthrenyl, anthracenyl or fluorenyl, and examples of heteroaromatic C₄-C₂₄-aryl in which no, one, two or three skeleton carbon atoms per cycle, but at least one skeleton carbon atom in the entire molecule, may be substituted by heteroatoms selected from the group of nitrogen, sulphur or oxygen are, for example, pyridinyl, oxazolyl, benzofuranyl, dibenzofuranyl or quinolinyl.

Arylalkyl is in each case independently a straight-chain, cyclic, branched or unbranched alkyl radical as defined above, which may be substituted singly, multiply or fully by aryl radicals as defined above.

C₅-C₁₈-Arylalkyl is, for example, benzyl or (R)-or (S)-1-phenylethyl.

Halogen may be fluorine, chlorine, bromine or iodine, preferably fluorine, chlorine or bromine.

The carbon atom indicated by * in the general formula (I) or (II) is an asymmetric carbon atom which can have (R) or (S) configuration. In the process, according to the invention, there is inversion of configuration at the carbon atom indicated by *.

In the context of the invention, enantiomerically enriched compound of the general formula (I) or (II) means the enantiomerically pure compounds of the general formula (I) or (II) in (R) or (S) configuration or mixtures of the two particular enantiomers in which one enantiomer is present in an enantiomeric excess, also referred to below as ee, in comparison to the other enantiomer. This enantiomeric excess is preferably 2 to 100% ee, more preferably at least 60% ee and most preferably at least 85% ee. In preferred embodiments, this enantiomeric excess is at least 95% ee. A definition of the ee value is specified in the examples of this application.

Possible substituents of the R¹ to R⁵ radicals are substituents which behave very substantially inertly in the process according to the invention. These are, for example, alkoxy groups, carboxylic acid derivatives such as carboxylic esters, carboxamides, carboximides, carbonyl fluorides, carbonyl chlorides, carbonyl bromides and carboxylates, nitrile groups or fluorine groups.

In the context of the invention, all radical definitions, parameters and illustrations, above and listed below, in general or within areas of preference, may be combined with one another in any desired manner, i.e. also between the particular areas and areas of preference.

In the context of the invention, R¹ is more preferably an optionally substituted C₁-C₆-alkyl radical, in particular methyl.

In the context of the invention, X is more preferably —OR⁴ in which R⁴ is an optionally substituted C₁-C₆-alkyl radical, in particular methyl.

In a preferred embodiment of the process according to the invention, the enantiomerically enriched 2-fluorocarboyxlic ester of the general formula (I) is methyl (R)-2-fluoropropionate.

In the context of the invention, R⁵ is more preferably an optionally substituted C₁-C₆-alkyl radical or an optionally substituted C₆-C₂₄-aryl radical, in particular methyl, trifluoromethyl or p-tolyl.

In a preferred embodiment of the process according to the invention, the sulphonic ester of the general formula (II) is methyl (S)-2-methanesulphonyloxypropionate.

The enantiomerically enriched sulphonic esters of the general formula (II) may be prepared in a manner known per se by deamminating the corresponding enantiomerically enriched 2-aminocarboxylic acids with sodium nitrite in dilute acid. The deamination is described, for example, in M. Winitz et al.; J. Am. Chem. Soc. 1956, 78, 2423. The methyl (S)-2-mesyloxypropionate used as the starting material in a preferred embodiment may be prepared readily according to a literature method (e.g. Fritz-Langhals, E.; Schütz, G.; Tetrahedron Letters, 1993, 34, 293-296) from commercially available methyl (S)-lactate without racemization. Commercially available methyl (S)-lactate typically has an optical purity of 97%. For the methyl (S)-2-methanesulphonyloxypropionate used in the process, this likewise results in an optical purity of 97%.

The solvents used are preferably one or more carboxamides. Suitable carboxamides are, for example, those of the general formula (II)
R⁶—C(O)NR⁷R⁸ (III)
in which
R⁶, R⁷ and R⁸ are each independently H, an optionally substituted C₁-C₁₈-alkyl radical, preferably an optionally substituted C₁-C₆-alkyl radical, or an optionally substituted C₄-C₂₄-aryl radical, preferably an optionally substituted C₆-C₂₄-aryl radical.

Examples of such carboxamides include formamide, N-methylformamide, N,N-diemthylformamide, acetamide, N-methylacetamide or N,N-dimethylacetamide. Preferred carboxamides are formamide, N-methylformamide, acetamide or N-methylacetamide; particular preference is given to formamide.

In the context of the invention, the solvent comprising at least one carboxamide may also comprise further solvent components, for example water, alcohols such as methanol, ethanol, n-propanol, i-propyl alcohol, ethers such as dioxane, tetrahydrofuran, diethyl ether, diethylene glycol dimethyl ether, chlorinated aliphatic hydrocarbons such as dichloromethane, chloroform, carbon tetrachloride, 1,2-dichloromethane, trichloroethylene, aliphatic or aromatic hydrocarbons such as n-pentane, n-hexane, hexane isomer mixtures, n-heptane, n-octane, wash benzene, petroleum ether, benzene, toluene, xylenes, ketones such as acetone, methyl ethyl ketone, methyl i-butyl ketone, nitrobenzene or mixtures thereof. However, the solvent more preferably contains at least one carboxamide in an amount of at least 20%, more preferably at least 70%. In preferred embodiments, essentially none of the further solvent components listed above is present in the solvent comprising at least one carboxamide.

The process according to the invention is carried out in the presence of an alkali metal fluoride or tetraalkylammonium fluoride. In the context of the invention, the alkali metal fluoride or tetraalkylammonium fluoride used may also be mixtures of two or more alkali metal fluorides or tetraalkylammonium fluorides. Particular suitable alkali metal fluorides or tetraalkylammonium fluorides are, for example, potassium fluoride, caesium fluoride or tetrabutylammonium fluoride.

The mixture of at least one alkali metal fluoride or tetraalkylammonium fluoride and the solvent comprising at least one carboxamide should preferably contain at least 1.5 equivalents of the alkali metal fluoride or tetraalkylammonium fluoride based on the initially charged amount of the sulphonic ester of the general formula (II). At least 1.5 but less than 4.0 equivalents of the alkali metal fluoride or tetraalkylammonium fluoride based on the initially charged amount of the sulphonic ester of the general formula (II) should more preferably be present in the mixture. In preferred embodiments, 1.5 to 2.5 equivalents, in very preferred embodiments 1.8 to 2.5 equivalents, of the alkali metal fluoride or tetraalkylammonium fluoride, based on the initially charged amount of the sulphonic ester of the general formula (II), are present in the mixture.

The concentration of the alkali metal fluoride or tetraalkylammonium fluoride in the mixture of at least one alkali metal fluoride or tetraalkylammonium fluoride and the solvent comprising at least one carboxamide should preferably be at least 1.5 mol per litre.

The mixture of at least alkali metal fluoride or tetraalkylammonium fluoride and the solvent comprising at least one carboxamide is preferably prepared in such a way that the solid alkali metal fluoride(s) or tetraalkylammonium fluoride(s) is/are fully or partly dissolved and/or suspended in the solvent comprising at least one carboxamide.

In the context of the invention, the term solvent does not mean that all reaction components necessarily have to dissolve in it. The reaction may also be carried out in a suspension or emulsion of one or more reaction partners. The reaction may also be carried out in a solvent mixture having a miscibility gap, in which case at least one reaction partner in each case is partly or fully soluble in each phase of the mixture.

In a preferred embodiment of the process according to the invention, the mixture of at least one alkali metal fluoride or tetraalkylammonium fluoride and the solvent comprising at least one carboxamide is heated beforehand to a temperature of at least 50° C. and then subsequently added as a heated mixture to the initially charged sulphonic ester of the general formula (II). There may be preference for addition of the mixture in portions over a single full addition. The addition in portions may be effected within a few minutes up to several hours. In preferred embodiments, the mixture is added to the initially charged sulphonic ester of the general formula (II) within a time of 30 minutes to 4 hours. The addition is effected preferably at a temperature of at least 50° C., more preferably of 50 to 100° C., most preferably of 70 to 85° C.

After full addition of the mixture of at least one alkali metal fluoride or tetraalkylammonium fluoride and the solvent comprising at least one carboxamide to the initially charged sulphonic ester of the general formula (II), it may be advantageous to stir the reaction mixture further at the same temperature and the same pressure for a period of a few minutes to several hours. Preference is given to doing this until there is virtually full conversion of the initially charged sulphonic ester of the general formula (II). In preferred embodiments, the reaction mixture is stirred further at the same temperature and the same pressure for at least 30 minutes, preferably 1 to 5 hours, more preferably 1 to 2 hours, in order to achieve a maximum yield.

The mixture of at least one alkali metal fluoride or tetraalkylammonium fluoride and the solvent comprising at least one carboxamide should preferably not contain any water before the addition to the initially charged sulphonic ester of the general formula (II). This may be achieved, for example, by dissolving the appropriate amount of anhydrous fluoride in anhydrous solvent under protective gas. However, it is also possible to use hydrous fluoride and solvent in technical-grade quality when the water present is very substantially entrained out after the preparation of the mixture by distilling off a portion of the solvent. For example, in the case of use of technical-grade formamide as the solvent and hydrous potassium fluoride, the water present may be removed very substantially from the mixture by distilling off approx. 5 to 10% of the amount of formamide.

In addition, it may be advantageous when the enantiomerically enriched 2-fluorocarboxylic ester of the general formula (I) formed is removed very rapidly from the reaction mixture. This may be done, for example, by continuously distilling off during the reaction. This allows both yield and optical purity of the product to be increased. In a preferred embodiment of the process according to the invention, the enantiomerically enriched 2-fluorocarboxylic ester of the general formula (I) formed is condensed during the reaction continuously into a cooled receiver at a pressure of less than 1 bar. Particularly advantageous in this context are a short path between reaction vessel and cooled receiver, and a very low temperature in this cooled receiver. The cooled receiver should preferably be arranged in such a way that the product does not run back into the reaction vessel after the condensation. However, the enantiomerically enriched 2-fluorocarboxylic ester of the general formula (I) formed may also be isolated only after the end of the reaction. This may likewise be done by distilling-off or condensing into a cooled receiver.

The enantiomerically enriched 2-fluorocarboxylic ester of the general formula (I), in particular methyl (R)-2-fluoropropionate, prepared by the process according to the invention may optionally be further purified by a fine distillation with appropriate column. In general, this is, though, not necessary, since the product, after the end of the reaction, can be isolated from the cooled receiver with a chemical purity of up to 99%.

The examples which follow serve to illustrate and demonstrate the invention by way of example, but do not constitute any restriction.

### EXAMPLES

In all of the examples which follow, the optical purity of the compounds of the general formula (I) or (II) with (R) or (S) configuration was determined by chromatographic methods (GC or HPLC) on a chiral column material and reported using the ee (enantiomeric excess) (S) or (R) value defined below.

The ee value is calculated by the following formulae:

$$ee ( S ) = \frac{m ( S ) - m ( R )}{m ( S + R )} ⨯ 100 \%$$

$$ee ( R ) = \frac{m ( R ) - m ( S )}{m ( S + R )} ⨯ 100 \%$$

where ee(S) and ee(R) are the optical purity of the S and R enantiomer respectively, m(S) is the amount of the S enantiomer and m(R) is the amount of the R enantiomer. (Examples: for a racemate: R=S=>ee=0; for the pure (S) form: ee(S)=100%; for a ratio of S:R=9:1, ee(S)=80%)

### Example 1

Approx. 50 ml of formamide were distilled off at 110° C. and 20 mbar from a mixture of 18.59 g (0.32 mol; 2.0 equivalents) of potassium fluoride and 200 ml of formamide. The thus obtained solution was then cooled to 80° C. under argon and charged into a jacketed dropping funnel heated to 80° C. Under argon, a 250 ml three-necked flask was initially charged with 30.49 g (0.16 mol; 1.0 equivalent; 95.6%; 97% ee) of methyl (S)-2-mesyloxypropionate and heated to 80° C. at 20 mbar. Subsequently, the potassium fluoride solution at 80° C. was added dropwise to the methyl (S)-2-mesyloxypropionate within one hour. The methyl (R)-2-fluoropropionate formed was collected continuously in a cold trap cooled to −78° C., connected to the reaction flask. After the potassium fluoride solution had been added completely, the mixture was stirred at 80° C. and 20 mbar for a further hour. From the cold trap, it was possible after thawing to isolate 12.5 g (73% yield) of methyl (R)-2-fluoropropionate with a chemical purity of 99% and an optical purity of 97%.

### Example 2

Approx. 50 ml of formamide were distilled off at 110° C. and 20 mbar from a mixture of 13.94 g (0.24 mol; 1.5 equivalents) of potassium fluoride and 250 ml of formamide. The thus obtained solution was then cooled to 80° C. under argon and charged into a jacketed dropping funnel heated to 80° C. Under argon, a 250 ml three-necked flask was initially charged with 30.49 g (0.16 mol; 1.0 equivalent; 95.6%; 97% ee) of methyl (S)-2-mesyloxypropionate and heated to 80° C. at 20 mbar. Subsequently, the potassium fluoride solution at 80° C. was added dropwise to the methyl (S)-2-mesyloxypropionate within one hour. The methyl (R)-2-fluoropropionate formed was collected continuously in a cold trap cooled to −78° C., connected to the reaction flask. After the potassium fluoride solution had been added completely, the mixture was stirred at 80° C. and 20 mbar for a further hour. From the cold trap, it was possible after thawing to isolate 12.0 g (64% yield) of methyl (R)-2-fluoropropionate with a chemical purity of 90% and an optical purity of 97%.

### Example 3

Approx. 50 ml of formamide were distilled off at 110° C. and 20 mbar from a mixture of 13.94 g (0.24 mol; 1.5 equivalents) of potassium fluoride and 200 ml of formamide. The thus obtained solution was then cooled to 50° C. under argon and charged into a jacketed dropping funnel heated to 50° C. Under argon, a 250 ml three-necked flask was initially charged with 30.49 g (0.16 mol; 1.0 equivalent; 95.6%; 97% ee) of methyl (S)-2-mesyloxypropionate and heated to 50° C. at 20 mbar. Subsequently, the potassium fluoride solution at 50° C. was added dropwise to the methyl (S)-2-mesyloxypropionate within one hour. The methyl (R)-2-fluoropropionate formed was collected continuously in a cold trap cooled to −78° C., connected to the reaction flask. After the potassium fluoride solution had been added completely, the mixture was stirred at 50° C. and 20 mbar for a further hour. From the cold trap, it was possible after thawing to isolate 1.9 g (9% yield) of methyl (R)-2-fluoropropionate with a chemical purity of 78% and an optical purity of 97%.

1. Process for preparing an enantiomerically enriched 2-fluorocarboxylic ester of the general formula (I) 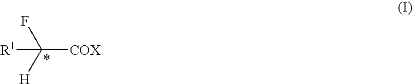
in which
R₁ is an optionally substituted C₁-C₁₈-alkyl radical, an optionally substituted C₄-C₂₄-aryl radical or an optionally substituted C₅-C₁₈-arylalkyl radical and
X is halogen, —NR²R³ or —OR⁴, in which R², R³ and R⁴ are each independently H or an optionally substituted C₁-C₁₈-alkyl radical, an optionally substituted C₄-C₂₄-aryl radical or an optionally substituted C₅-C₁₈-arylalkyl radical, or R² and R³ together are an optionally substituted C₁-C₁₈-alkylene radical, an optionally substituted C₄-C₂₄-arylene radical or an optionally substituted C₅-C₁₈-arylalkylene radical,
by reacting an enantiomerically enriched sulphonic ester of the general formula (II) 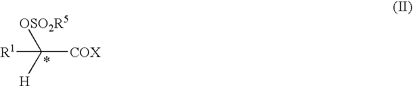
in which
R⁵ is an optionally substituted C₁-C₁₈-alkyl radical, an optionally substituted C₄-C₂₄-aryl radical or an optionally substituted C₅-C₁₈-arylalkyl radical and
R¹ and X are each as defined for the general formula (I)
with at least one alkali metal fluoride or tetraalkylammonium fluoride in a solvent comprising at least one carboxamide, characterized in that the sulphonic ester of the general formula (II) is initially charged and a mixture of at least one alkali metal fluoride or tetraalkylammonium fluoride and at least one carboxamide is subsequently added.

2. Process according to claim 1, characterized in that the sulphonic ester of the general formula (II) is initially charged at a pressure of less than 1 bar.

3. Process according to claim 1, characterized in that the sulphonic ester of the general formula (II) is initially charged at a temperature of at least 50° C.

4. Process according to claim 1, characterized in that R¹ is an optionally substituted C₁-C₆-alkyl radical.

5. Process according to claim 1, characterized in that X is —OR⁴ in which R⁴ is an optionally substituted C₁-C₆-alkyl radical.

6. Process according to claim 1, characterized in that the enantiomerically enriched 2-fluorocarboxylic ester of the general formula (I) is methyl (R)-2-fluoropropionate.

7. Process according to claim 1, characterized in that R⁵ is an optionally substituted C₁-C₆-alkyl radical or an optionally substituted C₁-C₂₄-aryl radical.

8. Process according to claim 1, characterized in that the sulphonic ester of the general formula (II) is methyl (S)-2-methanesulphonyloxypropionate.

9. Process according to claim 1, characterized in that the carboxamide is selected from formamide, N-methylformamide, N,N-dimethylformamide, acetamide, N-methylacetamide or N,N-dimethylacetamide.

10. Process according to claim 1, characterized in that the alkali metal fluoride or tetraalkylammonium fluoride is potassium fluoride, caesium fluoride or tetrabutylammonium fluoride.

11. Process according to claim 1, characterized in that the mixture of at least one alkali metal fluoride or tetraalkylammonium fluoride and the solvent comprising at least one carboxamide contains at least 1.5 equivalents of the alkali metal fluoride of tetraalkylammonium fluoride based on the initially charged amount of the sulphonic ester of the general formula (II).

12. Process according to claim 1, characterized in that the mixture of at least one alkali metal fluoride or tetraalkylammonium fluoride and the solvent comprising at least one carboxamide has a concentration of at least 1.5 mol of alkali metal fluoride or tetraalkylammonium fluoride per litre.

13. Process according to claim 1, characterized in that the mixture of at least one alkali metal fluoride or tetraalkylammonium fluoride and the solvent comprising at least one carboxamide is heated to a temperature of at least 50° C. and then added, as a heated mixture, to the initially charged sulphonic ester of the general formula (II).

14. Process according to claim 1, characterized in that the enantiomerically enriched 2-fluorocarboxylic ester of the general formula (I) formed is condensed during the reaction at a pressure of less than 1 bar continuously into a cooled receiver.
